(19) United States
(12) Patent Application Publication
(10) Pub. No.: US 2002/0091443 A1
(43) Pub. Date: Jul. 11, 2002

(54) PROSTHETIC IMPLANT FOR BODY AUGMENTATION, METHODS OF AUGMENTING SHAPE OR APPEARANCE OF A BODY AND AN ENDOSCOPIC PROSTHETIC IMPLANT INSTRUMENT

(76) Inventor: INBAE YOON, PHOENIX, MD (US)

Correspondence Address: BLANK ROME COMISKY & MCCAULEY LLP, THE FARRAGUT BUILDING SUITE 1000, 900 17TH STREET NW, WASHINGTON, DC, 20006, US

(21) Appl. No.: 09/271,915

(22) Filed: Mar. 18, 1999

(51) Int. Cl. A61F 2/12

(52) U.S. Cl. 623/7

(57) ABSTRACT

A prosthetic implant for use in augmentation of the shape of a human body or replacement of animal or human body tissue includes an expandable body member having a first predetermined shape and a first volume in an expanded condition and a second predetermined shape in a second volume smaller than the first volume in a compressed condition. An endoscopic prosthetic implant instrument inserts the prosthetic implant through an anatomical wall of a body and includes a hollow sleeve member, a pusher member and a clam shell mechanism. A combination endoscope and prosthetic implant instrument includes an endoscope having an operating channel that slidably receives the endoscopic prosthetic implant instrument. A method using the prosthetic implant augments the shape or appearance of a human body or replacement of animal or human body tissue. Another method uses the prosthetic implant along with the endoscopic prosthetic implant instrument to augment the shape or appearance of a human body or replacement of animal or body tissue.

### FIELD OF THE INVENTION

The invention relates to a prosthetic implant and method of using the prosthetic implant. More specifically, the invention is directed to a prosthetic implant and a method for augmenting the shape or appearance of a human body or replacement of animal or body tissue.

### BACKGROUND OF THE INVENTION

Reconstructive or cosmetic surgery has been practiced by healthcare professionals for decades. Generally, reconstructive and cosmetic surgery is used to recapture or enhance the shape or appearance of a person's body. Certain types of reconstructive or cosmetic surgery uses prosthetic implants. Specifically, prosthetic implants have been used for changing the shape or appearance of human body parts such as female breasts, testes, chin, cheek, pectoral muscles, and calf muscles. Generally, the prosthetic implant is a soft, silicone membrane filled with either a soft silicone gel having a consistency of gelatin or a sterile saline solution.

Augmentation mammoplasty, also referred to as “breast augmentation,” is sometimes elected by women to enlarge small or underdeveloped breasts. Thus, breast implants add volume to the total breast tissue.

The breast implant is placed either under breast tissue, commonly referred to as subglandular augmentation, or under the pectoralis muscle, commonly referred to as submuscular augmentation. If the breast implant is the type that requires filling by saline solution, the soft silicone membrane is usually inserted in an empty state behind fatty tissue of the breast. Once properly positioned, the silicone membrane is then filled with the appropriate amount of saline solution to provide a desired shape and size of the augmented breast. Using an empty silicone membrane enables its insertion through a small incision inconspicuously made in the breast fold, under the arm or around the nipple. However, a “fill kit” is necessary to fill the empty silicone membrane with sterile saline or gel. The kit includes a syringe, tubing, connectors and a container of saline or gel. Also, the silicone membrane typically has a valve through which the saline or gel is injected.

Alternatively, the breast implant can be implanted by open surgery. Using open surgery, the breast implant no longer requires a valve in order to inject the saline or gel because the silicone membrane is prefilled with saline or gelatin. However, the incision is made significantly larger to facilitate placement of the prefilled silicone membrane in the breast. Further, the larger incision may scar and become aesthetically undesirable.

Concerns have been raised regarding the safety of silicone gel filling materials for prosthetic breast implants. To address these concerns, other types of prosthetic implants using alternative fill materials have been used. For example, in U.S. Pat. No. 5,658,329, one of several implant filling materials is polyvinyl alcohol. U.S. Pat. No. 4,731,081 teaches an implantable rupture-resistant inflatable mammary prosthesis that also uses polyvinyl alcohol as the fill material.

It would be beneficial to provide a prosthetic implant for body augmentation that does not require filling a membrane and can be inserted into the body quickly and easily and, preferably, by a method that uses a small incision.

### OBJECTS AND SUMMARY OF THE INVENTION

An object of the invention is to provide a method of body augmentation and a prosthetic implant that does not require filling a membrane either before or after insertion of the prosthetic implant.

Another object of the invention is to provide a prosthetic implant that can be inserted into the body by either endoscopic or open surgery.

Accordingly, a method of body augmentation and a prosthetic implant of the invention are hereinafter described. The prosthetic implant of the invention is used for augmentation of the shape of a human body or for replacement of animal or human body tissue. The prosthetic implant of the invention includes an expandable body member having an expanded condition and a compressed condition. In the expanded condition, the prosthetic implant of the invention assumes a first predetermined shape and has a first volume. In the compressed condition, the expandable body member assumes a second predetermined shape and has a second volume. The second volume is smaller than the first volume when the expandable body member is in the compressed condition.

The method of the invention augments the shape or appearance of a human body or replaces animal or human body tissue. The method includes the steps of providing the expandable body member as described above, piercing an anatomical wall of the body to form an opening therethrough, inserting the expandable member in its compressed condition through the opening in the anatomical wall to a desired position in the body and expanding the expandable member to its expanded condition thereby augmenting the shape or appearance of the body.

### BRIEF DESCRIPTION OF THE DRAWINGS

FIG. 1 is a perspective view of an upper torso of a female human body illustrating a prosthetic implant of the invention augmenting a breast.

FIG. 2 is an elevational view of the prosthetic implant of the invention in an expanded condition.

FIG. 3 is an elevational view of the prosthetic implant of the invention in a compressed condition.

FIG. 4 is a cross-sectional view of a normal female breast.

FIG. 5 is a cross-sectional view with the prosthetic implant of the invention disposed within the female breast between the pectoralis muscle and glandular breast tissue.

FIG. 6 is a cross-sectional view of the female breast with the prosthetic implant of the invention disposed behind the pectoralis muscle.

FIG. 7 is a perspective view of the prosthetic implant in the compressed condition.

FIG. 8 is a partially broken, side elevational view of a prosthetic implant instrument.

FIG. 9 is a cross-sectional view of a distal portion of the prosthetic implant instrument with the prosthetic implant of the invention in the compressed condition disposed therein.

FIG. 10 is a cross-sectional view of the distal and of the prosthetic implant instrument illustrating the prosthetic implant being extracted therefrom.

FIG. 11 is a cross-sectional view of the prosthetic implant of the present invention in the expanded condition.

FIG. 12 is a cross-sectional view of another alternative embodiment of the prosthetic implant device of the invention having a plurality of spines disposed therein.

FIG. 13 is a cross-sectional view of an alternative embodiment of the prosthetic implant of the invention encapsulated in a membrane.

FIGS. 14A and 14B are cross-sectional views of another alternative embodiment of the implant device of the invention in the expanded condition and compressed condition respectively with the membrane having a thickened portion.

FIGS. 15A and 15B are cross-sectional views of another alternative embodiment of the prosthetic implant of the invention in the expanded condition and compressed condition respectively with the membrane having a valve assembly integrally formed therewith.

FIG. 16 is a perspective view of the prosthetic implant instrument disposed near a wrinkled face of a human.

FIG. 17 is a side view showing a distal end of the prosthetic implant instrument positioned behind the wrinkled cheek of the human in FIG. 16.

FIG. 18 is a side view of the prosthetic implant instrument behind the wrinkled cheek of the human in FIG. 16 with the prosthetic implant in the compressed condition being extracted therefrom.

FIG. 19 is a side elevational view illustrating the prosthetic implant in the expanded condition stretching the wrinkled cheek of the human.

FIG. 20 is a perspective view of the expanded prosthetic implant shown in FIG. 19.

FIG. 21 is a perspective view of the prosthetic implant instrument slidably disposed in an endoscope.

### DETAILED DESCRIPTION OF THE EXEMPLARY EMBODIMENTS

A method of augmentation of the shape or appearance of a body, i.e., an animal or human body, and a prosthetic implant of the invention are hereinafter described. The method and the prosthetic implant of the invention are described by way of example using female breast augmentation. However, one of ordinary skill in the art would appreciate that the method and prosthetic implant of the invention can be used for other types of reconstructive or cosmetic surgeries, such as testicular prostheses as well as chin, cheek, pectoral, buttocks and calf enhancement. Also, the method and prosthetic implant of the invention can be used for smoothing wrinkles of a person's face, forehead, neck or other body parts.

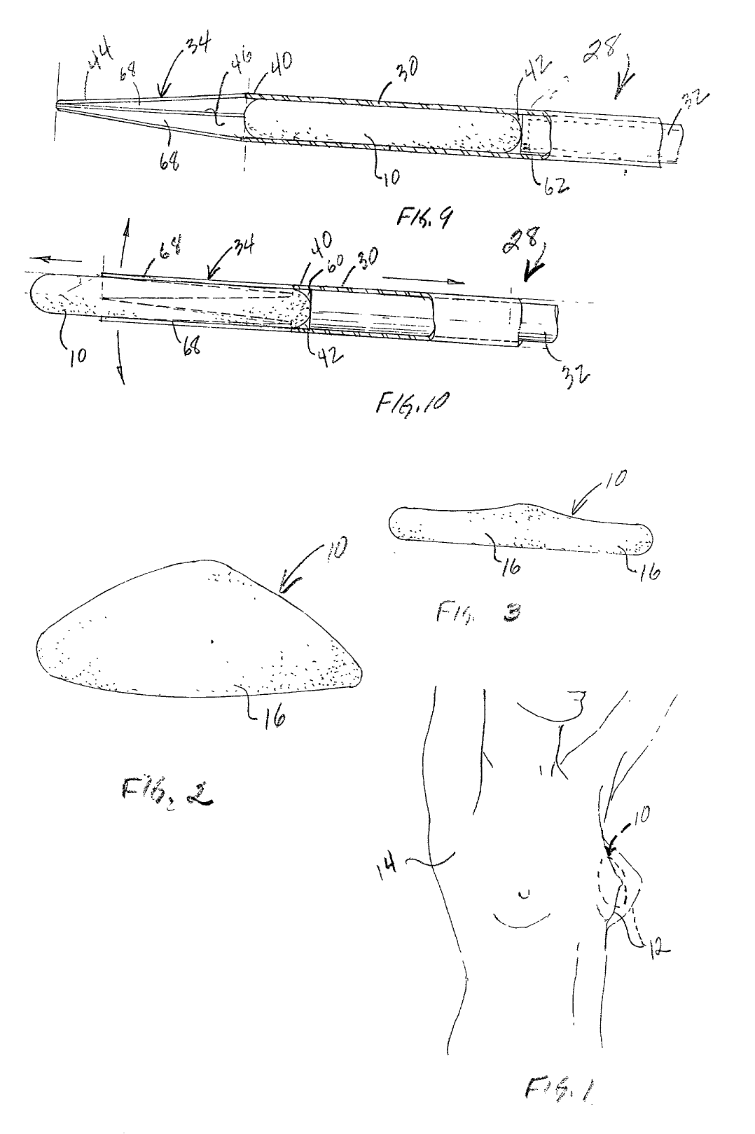

A prosthetic implant 10 of the present invention is generally introduced in FIGS. 1-7. By way of example and not by limitation, the prosthetic implant 10 of the invention is used for augmentation of a breast 12 of a female human body 14 as shown in FIG. 1. Such augmentation can be applied to modify or configure a shape of the breast 12 or, in the event of a radical mastectomy, to replace the human body tissue of the breast 12. Furthermore, augmentation can be used for animals as well as human beings.

As best shown in FIGS. 2 and 3, the prosthetic implant includes an expandable body member 16 fabricated from a sponge material. Preferably, the sponge material is polyvinyl alcohol or other sponge material that is medically acceptable and non-reactive to living tissue. In FIG. 2, the expandable body member 16 is in an expanded condition while, in FIG. 3, the expandable body member 16 is in has a compressed condition. In the expanded condition, the expandable body member 16 has a first predetermined shape and a first volume as shown by example in FIG. 2 as a generally conical shape. In the compressed condition, the expandable body member 16 has a second predetermined shape and a second volume as shown by example in FIG. 3 as a generally rod-like shape. By comparison, the second volume is smaller than the first volume. As described in more detail below, the expandable body member 16 in the compressed condition facilitates insertion into the breast 12. In the compressed condition, the sponge material of the prosthetic implant 10 of the invention is dehydrated. In the expanded condition, the sponge material of the prosthetic implant 10 is hydrated. Hydrating the sponge material expands the expandable body member 16 from its compressed condition to its expanded condition. In other words, the expandable body member has the first predetermined shape and the first volume in a hydrated condition and has the second predetermined shape and the second volume in a dehydrated condition. Preferably, hydrating the sponge material of the prosthetic implant 10 of the invention occurs with an aqueous or saline solution. However, hydrating the sponge material might also be achieved by absorption of the natural bodily fluids in the surrounding anatomical tissue.

In FIG. 4, the breast 12 is shown in cross-section prior to augmentation. The breast 12 is shown in cross-section as having skin 18 forming an anatomical wall that envelops glandular tissue 20. Disposed behind the glandular tissue 20 is a pectoralis muscle 22 and a rib cage 24 supporting the pectoralis muscle 22.

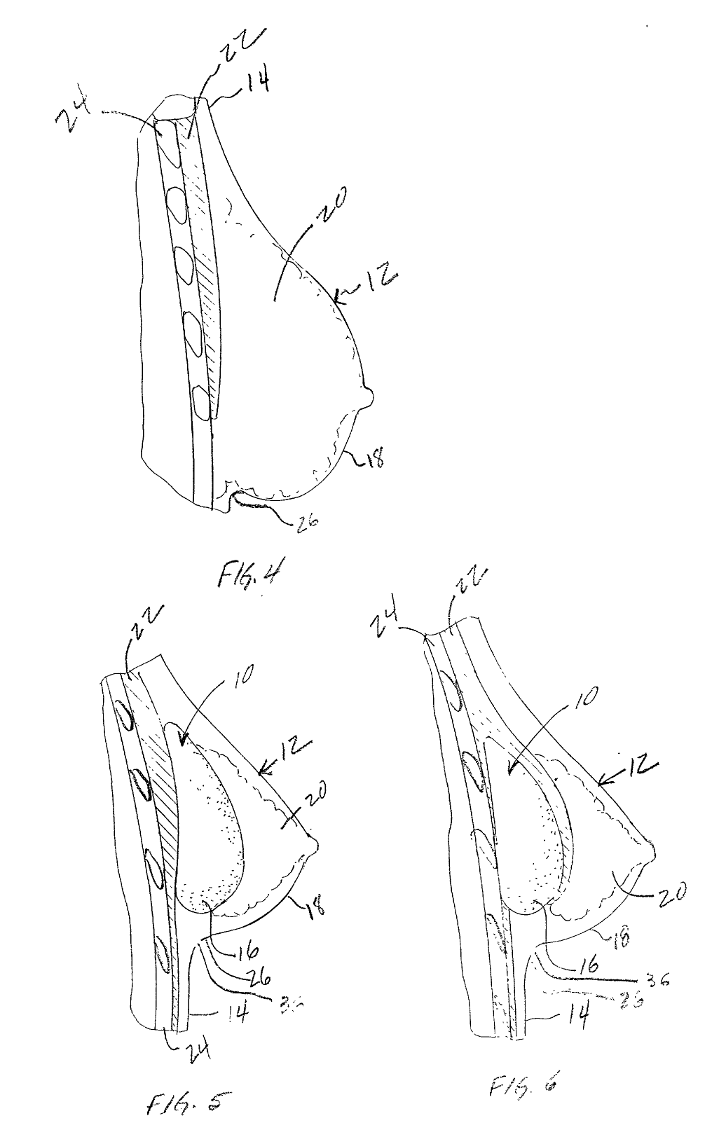

One method of inserting the prosthetic implant 10 of the invention into the breast 12 is by open surgery. For open surgery, a large incision is made at a breast fold 26 and the expandable body member 16 is inserted into the breast 12 as shown FIGS. 5 or 6 in the expanded or hydrated condition. In other words, the expandable sponge material is first hydrated exteriorly of the breast 12 before being implanted into the female human body 14. In FIG. 5, the prosthetic implant 10 of the invention is disposed between the glandular tissue 20 and the pectoralis muscle 22. In FIG. 6, the prosthetic implant 10 of the invention is inserted between the pectoralis muscle 22 and the rib cage 24.

Alternatively, the prosthetic implant 10 of the invention can be inserted into the breast endoscopically using, for example, an endoscopic prosthetic implant instrument 28 as shown in FIGS. 8-10. Using the endoscopic prosthetic implant instrument 28 requires only a minor incision at the breast fold 26 in order to insert the expandable body member 16 while in its compressed or dehydrated condition as shown in FIG. 7.

In FIGS. 8-10, the endoscopic prosthetic implant instrument 28 is used for inserting the prosthetic implant 10 through the skin 18 forming the anatomical wall of the human body 14. It includes a hollow sleeve member 30, a pusher member 32, and a clam shell mechanism 34.

The sleeve member 30 extends along a longitudinal axis A and is adapted for insertion into an incision or opening 36, as shown in FIGS. 5 and 6 in the anatomical wall, i.e., the skin 18, of the human body 14. The sleeve member 30 has an opened end 38 and an opposite distal end 40 and defines an interior channel 42 which is sized to slidably receive the prosthetic implant 10 preferably at the distal end 40.

The pusher member 32 extends along the longitudinal axis A and is disposed within the sleeve member 30. The pusher member 32 is sized to be received by the opened end 38 of the sleeve member 30. The pusher member 32 is also sized to simultaneously contact the prosthetic implant 10 and extend outwardly from the interior channel 42 as best shown in FIG. 8.

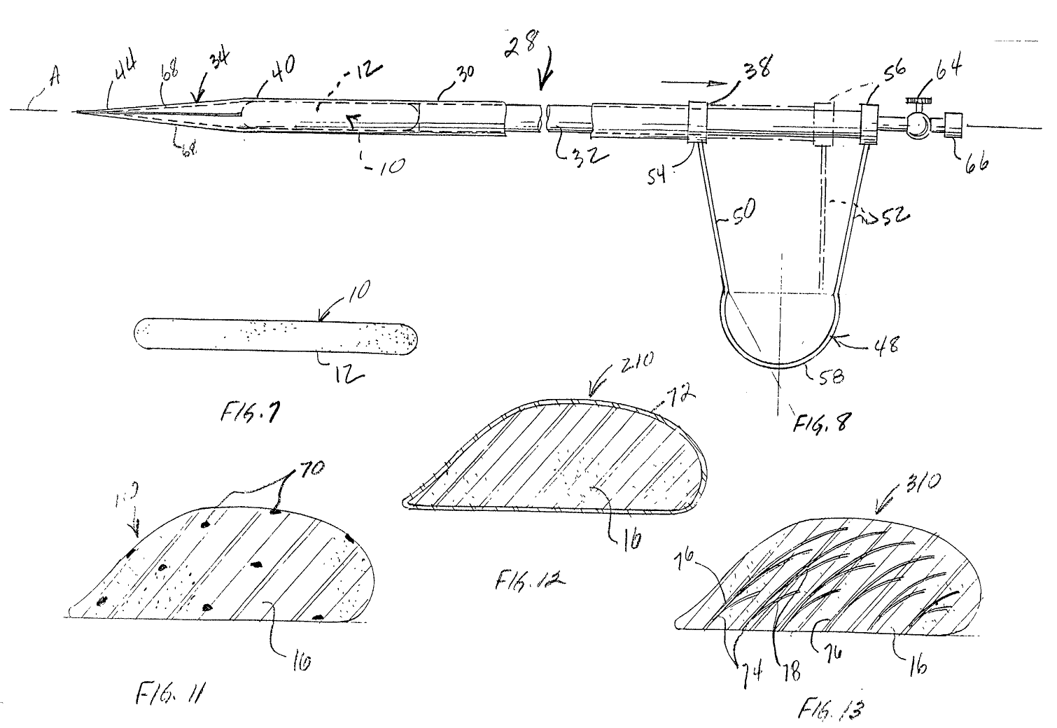

As illustrated in FIGS. 9 and 10, the clam shell mechanism 34 is hingably connected to the sleeve member 30 at its distal end 40. The clam shell mechanism 34 is operative to move to and between a closed state as shown in FIG. 9 and an opened state as shown in FIG. 10. In the closed state, the clam shell mechanism 34 forms a tip portion 44 as well as a conically-shaped interior cavity 46 which communicates with the interior channel 42. In FIG. 9, the clam shell mechanism 34 encloses the prosthetic implant 10 at the distal end 40 of the sleeve member 30 between the tip portion 44 of the clam shell mechanism 34 and the pusher member 32. In the opened state, the clam shell mechanism 34 permits the prosthetic implant 10 to exit the endoscopic prosthetic implant instrument 28. The prosthetic implant 10 exits the endoscopic prosthetic implant instrument 28 when the pusher member 32 which is in contact with the prosthetic implant 10 slides further into the sleeve member as shown sequentially in FIGS. 9 and 10. Thus, the pusher member 32 pushes the prosthetic implant 10 through the interior channel 42 and out of the clam shell mechanism 34.

As best shown in FIG. 8, the endoscopic prosthetic implant instrument 28 includes a handle structure 48 that has a sleeve handle portion 50 and a pusher handle portion 52. The sleeve handle portion 50 is connected to the sleeve member 30 adjacent the opened end 38. Particularly, a sleeve collar 54 is connected to the sleeve member 30 at the opened end 38 and the sleeve handle portion 50 is connected to the sleeve collar 54. Likewise, the pusher handle portion 52 is connected to the pusher member 32 at a location exteriorly of the interior channel 42. At this location, a pusher collar 56 is connected to the pusher member 32 which, in turn, is connected to the pusher handle portion 52.

The handle structure 48 also includes a biasing member 58 which biases the sleeve handle portion 50 and the pusher handle portion 52 away from one another. A skilled artisan would appreciate that squeezing the sleeve handle portion 50 and the pusher handle portion 52 together results in pushing the prosthetic implant 10 through the endoscopic prosthetic implant instrument 28. The squeezing action is illustrated in FIG. 8 by showing the pusher handle portion 52 and the pusher collar 56 being displaced from its original position (drawn phantomly). Although not by way of limitation, the biasing member 58 interconnects the sleeve handle portion 50 and the pusher handle portion 52 of the handle structure 48.

As shown in FIG. 9, the pusher member 32 is a hollow tube and has an abutting end 60 that contacts the prosthetic implant 10. The abutting end 60 has an orifice 62 formed therethrough. Also, as shown in FIG. 8, the pusher member 32 includes a valve assembly 64 and a connector 66. This arrangement selectively permits a hydrating liquid such as saline solution to flow through the pusher member 32 and out of the orifice 62 to hydrate the prosthetic implant 10, if desired. It is possible to hydrate the prosthetic implant when it is disposed within the endoscopic prosthetic implant instrument 28 or after it exits the endoscopic prosthetic implant instrument 28.

With reference to FIGS. 9 and 10, the clam shell mechanism 34 includes a plurality of clam shell segments 68. Each clam shell segment 68 is generally arcuately and triangularly shaped and is hingably connected to the distal end 40 of the sleeve member 30 in a resiliently biased manner. Because the clam shell segments 68 are hingably connected in a resiliently biased manner, the clam shell mechanism 34 is biased in the closed state to form a cone shape as shown in FIGS. 8 and 9. One of ordinary skill in the art would appreciate that when the pusher member 32 pushes the prosthetic implant 10 through the interior channel 42 and out of the clam shell mechanism 34, the prosthetic implant 10 is pushed into the interior cavity 46 which, in turn, causes the clam shell mechanism 34 to move from the closed state to the opened state.

FIGS. 11-15B illustrate alternative embodiments of the prosthetic implant of the invention. In FIG. 11, a prosthetic implant 110 is illustrated in cross section to show medicaments 70 impregnated into and on the surface of the expandable body member 16. The medicaments can be either anesthetic, antibiotic and/or antiseptic agents.

In FIG. 12, a prosthetic implant 210, includes a plurality of spines 74. The spines 74 are embedded in the expandable body member 16 in order to increase the stiffness thereof. One of ordinary skill in the art would appreciate that either one spine 74 or a plurality of spines 74 can be used. Further, although not by way of limitation, each spine 74 includes a main shaft 76 having a plurality of spine branches 78 extending therefrom.

In FIG. 13, a prosthetic implant 310 includes a stretchable membrane 72 that envelops the expandable body member 16. It is appreciated that the stretchable membrane 72 covers the expandable body member 16 in both the expanded and compressed conditions. Although not by way of limitation, the stretchable membrane 72 can be made of polyvinyl alcohol sheet material, silicone or the like.

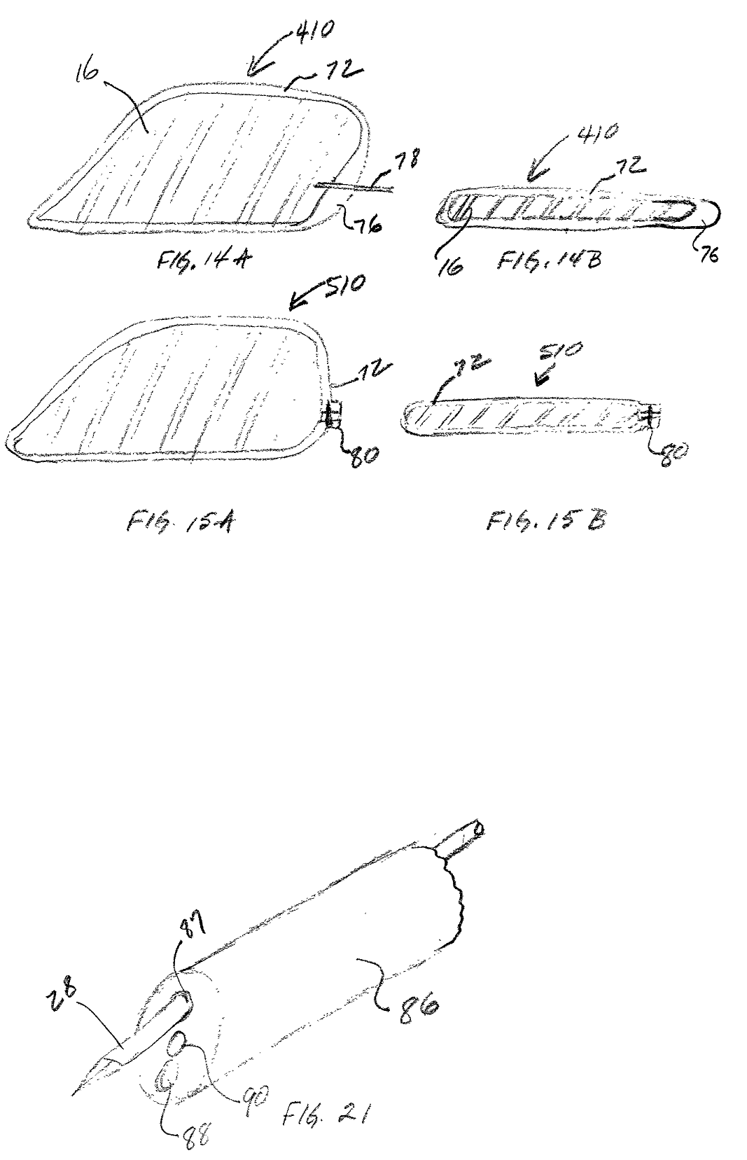

In FIGS. 14A and 14B, a prosthetic implant 410 is shown in the expanded condition and the compressed condition respectively. The stretchable membrane 72 includes a thickened portion 76. Once the prosthetic implant 410 is inserted at the desired position in the body, a needle 78, for example, penetrates the stretchable membrane 72 at the thickened portion 76. With the needle 78 extending into the membrane 72, a fluid such as the aqueous solution or a moisture-laden air can be injected into the membrane 72 to cause the expandable implant member 16 to expand from its compressed condition to its expanded condition. The needle 78 is then withdrawn from the stretchable membrane 72 and the thickened portion 76 collapses around an opening formed by the needle 78 to seal the fluid within the membrane. The thickened portion 76 of the membrane 72 is preferably integrally formed with the membrane. However, a skilled artisan would appreciate that the thickened portion 76 can be made from a material such as rubber and connected to the stretchable membrane 72 to achieve the same purpose.

In FIGS. 15A and 15B, a prosthetic implant 510 is shown in the expanded condition and the compressed condition respectively. The prosthetic implant 510 is similar to the prosthetic implant illustrating FIGS. 14A and 14B. However, in lieu of the thickened portion 76, a conventional valve assembly 80 is connected to the stretchable membrane 72. Likewise, the valve assembly 80 can be integrally formed with the stretchable membrane 72 or connected to it as a separate component.

A method for augmenting the shape or appearance of a human body using the prosthetic implants described above is explained. One of ordinary skill in the art would appreciate that the method of augmentation can also be used for the replacement of animal or human body tissue. The first step is providing the prosthetic implant of the invention as described above. The skin or anatomical wall 18 is punctured to form the incision or opening 36 through the anatomical wall. The prosthetic implant 10 is inserted in its compressed condition through the incision and into a desired position in the body. The prosthetic implant is then expanded to substantially its expanded condition. As described above, in the expanded condition the expandable body member assumes its first predetermined shape and its first predetermined volume thereby augmenting the shape or appearance of the body.

Using the endoscopic prosthetic implant instrument, for example, requires positioning the expandable body member in its compressed state into the tubular or hollow sleeve member. The hollow sleeve member is then inserted into the incision or opening in the anatomical wall and the expandable body member is then pushed out of the tubular or hollow sleeve member into the desired position in the body. The expandable body member in its compressed condition is then hydrated with an aqueous solution or by natural bodily fluids in order to expand it to the substantially expanded condition. Preferably, the expandable body member in its compressed condition is first positioned at the desired location in the body prior to hydrating it to its substantially expanded condition.

As shown in FIGS. 1 and 4-6, one desired position in the body is adjacent the breast 12 for augmenting the size and/or shape of the breast of the female human body 14. However, one of ordinary skill in the art would appreciate that augmentation may be performed on either a male or female body and on different anatomical body parts such as the buttocks for augmenting the shape of the buttocks. Further, augmentation using the prosthetic implant of the invention can be used adjacent wrinkled skin of the body to reduce or, possibly, eliminate the wrinkled appearance of the skin as illustrated in FIGS. 16-19.

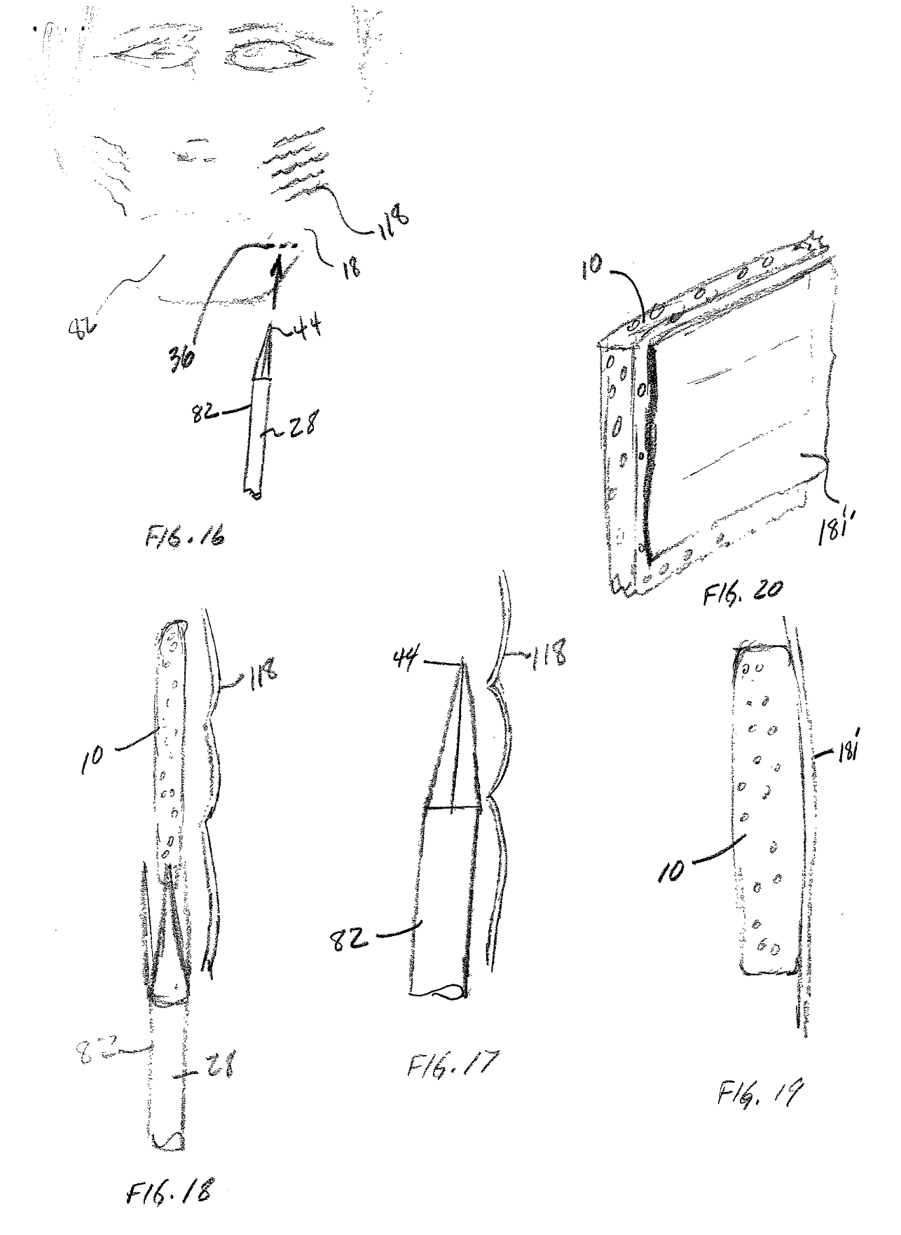

In FIG. 16, the endoscopic prosthetic implant instrument 28 is disposed adjacent a person's face 82 having wrinkled skin 118. A method of augmenting the appearance of the person's face 82 is described. The endoscopic prosthetic implant instrument 28 that contains a prosthetic implant in the compressed condition is placed near the wrinkled skin 118. The tip portion 44 of the endoscopic prosthetic implant instrument 28 is used to puncture the normal skin 18, i.e., the anatomical wall, to form the incision 36 (drawn phantomly). The incision 36 is located adjacent a position where the prosthetic implant is to be located. A distal portion 82 of the endoscopic prosthetic implant instrument is inserted through the incision 36 behind the wrinkled skin 118 as shown in FIGS. 17 and 18 at the position where the prosthetic implant 10 is to be located. The prosthetic implant 10 is then displaced from the endoscopic prosthetic implant instrument 28 at the position as best shown in FIG. 18. The distal portion 82 of the endoscopic prosthetic implant instrument 28 is withdrawn from behind the wrinkled skin 118 through the incision 36. As discussed above, the prosthetic implant 10 of the invention is expanded from the compressed condition (FIG. 18) to the expanded condition (FIGS. 19 and 20) to thereby augment the appearance of the wrinkled skin 118 of the person's face 82. As illustrated in FIGS. 19 and 20, the prosthetic implant 10 is expanded to its expanded condition to cause a slight pressure outwardly on the wrinkled skin to form a generally unwrinkled skin 118′.

In FIG. 21, the endoscopic prosthetic implant instrument 28 is received by a conventional endoscope 86 through an operating channel 87 sized to slidably receive the endoscopic prosthetic implant instrument 28. By way of example, the endoscope 86 includes an illumination device 88 and a viewing device 90. One of ordinary skill in the art would appreciate that using the conventional endoscope 86 along with the endoscopic prosthetic implant instrument 28 would enable the skilled artisan to guide the endoscopic prosthetic implant instrument 28 to a precise position after penetrating the incision. Further, one of ordinary skill in the art would comprehend that the conventional endoscope 86 could be either a rigid or a flexible endoscope and the viewing device 90 could use a conventional CMOS system, DMOS system, rod lens system, CCD system, fiber optic system, multiple lens system or the like.

The above-described embodiments and methods for augmentation of a body have been described by way of example only. One of ordinary skill in the art would appreciate that other modifications may be made to the embodiments without departing from the spirit and concept of the inventions.

What is claimed is:

1. A method of augmentation of the shape or appearance of a human body or replacement of animal or human body tissue having an anatomical wall, comprising the steps of:
providing an expandable body member having a first predetermined shape and a first volume in an expanded condition, said expandable body member having been compressed into a compressed condition having a second predetermined shape and a second volume smaller than the first volume;
piercing said anatomical wall to form an opening therethrough;
inserting said expandable body member in its compressed condition through the opening in said anatomical wall into a desired position in said body; and
expanding said expandable body member to substantially its expanded condition having its first predetermined shape and first predetermined volume to thereby augment the shape or appearance of the body.

2. The method of claim 1, wherein said inserting step comprises the steps of positioning the compressed expandable body member in a tubular member, inserting the tubular member into the opening in the anatomical wall and pushing said compressed expandable body member out of said tubular member into the desired position in said body.

3. The method of claim 1, wherein said expandable body member comprises a sponge material which is dehydrated in its compressed condition and hydrated in its expanded condition.

4. The method of claim 3, including the step of hydrating the expandable body member in its compressed condition with an aqueous solution to expand the expandable body member to substantially its expanded condition.

5. The method of claim 4, including the step of, prior to hydrating the expandable body member in its compressed condition, positioning the expandable body member in the desired position in the body.

6. The method of claim 3, wherein said sponge material is a polyvinyl alcohol sponge.

7. The method of claim 1, wherein said desired position in said body is adjacent the breast of a female for augmenting the size or shape of the breast of the female.

8. The method of claim 1, wherein said desired position in said body is adjacent the buttocks for augmenting the shape of the buttocks.

9. The method of claim 1, wherein said inserting step comprises the steps of positioning the compressed expandable body member adjacent wrinkled skin of the body to reduce the wrinkled appearance of the skin.

10. The method of claim 1, including the step of incorporating at least one of an anesthetic, antibiotic or antiseptic agent in said expandable body member.

11. A prosthetic implant for use in augmentation of the shape of a human body or replacement of human body tissue, comprising:
an expandable body member having a first predetermined shape and a first volume in an expanded condition and a second predetermined shape and a second volume smaller than the first volume in a compressed condition.

12. The prosthetic implant of claim 11, wherein the expandable body member has a first predetermined shape suitable for breast augmentation in a female body.

13. The prosthetic implant of claim 11, wherein the expandable body member comprises an expandable sponge material having said second predetermined shape and second volume in a dehydrated condition and said first predetermined shape and first volume in a hydrated condition.

14. The prosthetic implant of claim 13, wherein the expandable sponge material is a polyvinyl alcohol sponge.

15. The prosthetic implant of claim 11, including at least one of an anesthetic, antibiotic or antiseptic agent incorporated in said expandable body member.

16. The prosthetic implant of claim 11, including a stretchable membrane covering said expandable body member in its compressed condition and its expanded condition.

17. The prosthetic implant of claim 16, wherein the stretchable membrane is made of a polyvinyl alcohol sheet material.

18. The prosthetic implant of claim 11, including spine means embedded in said expandable body member for increasing the stiffness of said expandable body member.

19. The prosthetic implant of claim 11, wherein the expandable body member has a substantially cylindrical, rod-like shape in its compressed condition.

20. An endoscopic prosthetic implant instrument for inserting a prosthetic implant through an anatomical wall of a body, the endoscopic prosthetic implant instrument comprising:
a hollow sleeve member extending along a longitudinal axis and adapted for insertion into an opening formed in the anatomical wall of the body, the sleeve member having an opened end and an opposite distal end and defining an interior channel sized to slidably receive the prosthetic implant at least at the distal end;
a longitudinally-extending pusher member slidably disposed within the sleeve member and sized to be received by the opened end of the sleeve member and to simultaneously contact the prosthetic implant and extend outwardly from the interior channel; and
a clam shell mechanism hingably connected to the sleeve member at the distal end, the clam shell mechanism operative to move to and between a closed state wherein the clam shell mechanism forms a tip portion and an interior cavity in communication with the interior channel thereby enclosing the prosthetic implant at the distal end of the sleeve member between the tip portion and the pusher member and an opened state wherein the clam shell mechanism permits the prosthetic implant to exit the endoscopic prosthetic implant instrument when the pusher member in contact with the prosthetic implant slides into the sleeve member thereby pushing the prosthetic implant through the interior channel and out of the clam shell mechanism.

21. An endoscopic prosthetic implant instrument according to claim 20, further comprising a handle structure having a sleeve handle portion connected to the sleeve member adjacent the opened end and a pusher handle portion connected to the pusher member at a location exteriorly of the interior channel.

22. An endoscopic prosthetic implant instrument according to claim 21, wherein the handle structure includes a biasing member operative to bias the sleeve handle portion and the pusher handle portion away from one another.

23. An endoscopic prosthetic implant instrument according to claim 22, wherein the biasing member interconnects the sleeve handle portion and the pusher handle portion.

24. An endoscopic prosthetic implant instrument according to claim 20, wherein the pusher member is hollow.

25. An endoscopic prosthetic implant instrument according to claim 24, wherein the pusher member includes an abutting end that contacts the prosthetic implant, the abutting end has at least one orifice formed therethrough.

26. An endoscopic prosthetic implant instrument according to claim 25, wherein the pusher member includes a valve assembly.

27. An endoscopic prosthetic implant instrument according to claim 20, wherein the clam shell mechanism includes a plurality of clam shell segments, each clam shell segment being arcuately shaped and hingably connected to the distal end of the sleeve member in a resiliently biased manner such that the clam shell mechanism is biased in the closed state.

28. An endoscopic prosthetic implant instrument according to claim 27, wherein pushing the prosthetic implant through the interior channel and into the interior cavity causes the clam shell mechanism to move from the closed state to the opened state.

29. A method of augmentation of a shape or appearance of a human body or replacement of animal or human body tissue having an anatomical wall, comprising the steps of:
providing an endoscopic prosthetic implant instrument and a prosthetic implant removably disposed in the endoscopic prosthetic implant instrument in a compressed condition;
puncturing the anatomical wall with a tip portion of the endoscopic prosthetic implant instrument to form an incision located adjacent a position where the prosthetic implant is to be located;
inserting a distal portion of the endoscopic prosthetic implant behind the anatomical wall at the position where the prosthetic implant is to be located;
displacing the prosthetic implant from the endoscopic prosthetic implant instrument at the position;
withdrawing the distal portion of the endoscopic prosthetic implant instrument from behind the anatomical wall through the incision; and through the incision; and
expanding the prosthetic implant from the compressed condition to an expanded condition thereby augmenting the shape or appearance of the human body or replacing animal or human body tissue.

30. A combination endoscope and prosthetic implant instrument, comprising:
an endoscopic prosthetic implant instrument including a hollow sleeve member, a pusher member and a clam shell mechanism hingably connected to a distal end of the hollow sleeve member; and
an endoscope having an operating channel sized to slidably receive the endoscopic prosthetic implant instrument.
